(19) United States
(12) Patent Application Publication
(10) Pub. No.: US 2026/0157866 A1
(43) Pub. Date: Jun. 11, 2026

(54) ADJUSTABLE PROSTHETIC

(71) Applicant: Sia Reddy, Hartford, CT (US)

(72) Inventor: Sia Reddy, Hartford, CT (US)

(21) Appl. No.: 18/976,806

(22) Filed: Dec. 11, 2024

(51) Int. Cl. A61F 2/72; A61F 2/58; A61F 2/60; A61F 2/70

(52) U.S. Cl. CPC A61F 2/72; A61F 2/586; A61F 2002/607; A61F 2002/701

(57) ABSTRACT

In accordance with at least one aspect of this disclosure, a prosthetic includes a first portion extending along an axis configured to receive and partially surround a residual limb of a user. A second portion is moveably connected to the first portion, wherein the first portion and the second portion come together to define an interior space for supporting the prosthetic appendance on the residual limb of the user. A sensor is disposed on an inner surface of the first portion and within the interior space. A controller is operatively connected to the sensor to receive a first pressure signal from the sensor indicative of a pressure of the residual limb on the inner surface of the first portion. The controller is configured to adjust the second portion relative to the first portion based on the pressure signal received from the sensor to expand or retract a volume of the interior space of the first portion to accommodate radial growth and shrinkage of the residual limb relative to the axis.

### TECHNICAL FIELD

The present disclosure relates generally to prosthetics, and more particularly to adjustable prosthetics.

### BACKGROUND

Prosthetics and prosthetic limbs permit a person with only a residual limb to use the full functionality of the missing appendage, e.g., a hand or foot. Prosthetics are becoming more advanced in their ability to be configured for easy manipulation by the user. Current prosthetics, however, do not provide a means for adjusting to changes in the size of a person's residual limb throughout the day, or over an extended period of time to account for the growth of the wearer's residual limb. Currently available prosthetics can be incredibly expensive and can require frequent change to accommodate the growth of the limb.

Thus, there is an ever-present need for improved prosthetic devices. This disclosure provides a solution for this need.

### SUMMARY

In accordance with at least one aspect of this disclosure, a prosthetic includes a first portion extending along an axis configured to receive and partially surround a residual limb of a user. A second portion is moveably connected to the first portion, wherein the first portion and the second portion come together to define an interior space for supporting the prosthetic appendance on the residual limb of the user. A sensor is disposed on an inner surface of the first portion and within the interior space. A controller is operatively connected to the sensor to receive a first pressure signal from the sensor indicative of a pressure of the residual limb on the inner surface of the first portion. The controller is configured to adjust the second portion relative to the first portion based on the pressure signal received from the sensor to expand or retract a volume of the interior space of the first portion to accommodate radial growth and shrinkage of the residual limb relative to the axis.

In certain embodiments, the prosthetic can have a spool having a filament wound thereon. The second portion may be connected to the first portion by the filament. A motor (e.g., a servo) can be operatively connected to the spool to drive the spool. The controller can be configured to cause the motor to rotate the spool in a first direction to unwind the spool to expand the volume of the interior space of the first portion when the first pressure signal from the sensor is above a first threshold. Additionally, the controller can be configured to cause the motor to rotate the spool in a second direction to wind the spool to retract the volume of the interior space of the first portion when the first pressure signal from the sensor is below a second threshold.

In certain embodiments the prosthetic can comprise a circuit board that can be operatively connected to the sensor and the controller.

In certain embodiments the circuit board can be embedded within the first portion.

In certain embodiments the prosthetic can comprise a power source operatively connected to the circuit board and can be configured to power the controller and the sensor.

In certain embodiments the power source can include a rechargeable battery.

In certain embodiments the controller can be configured to convert the first pressure signal to a voltage signal. The first threshold can be a first voltage threshold and the second threshold can be a second voltage threshold different from the first voltage threshold.

In certain embodiments the prosthetic can have a coupling portion defined at a first end of the first portion and can be configured and adapted to receive and couple an appendage to the first portion.

In certain embodiments the prosthetic can have a strap that can be operatively connected to a second end of the first portion that can be configured to secure the prosthetic to the residual limb.

In certain embodiments the prosthetic can have at least one digit-like structure that can be hingedly coupled to the coupling portion.

In certain embodiments the prosthetic can have a digit sensor disposed on a tip of the at least one digit-like structure that can be operatively connected to the controller. The controller can be configured to receive a second pressure signal from the digit sensor indicative of a pressure on the at least one digit-like structure. A vibration motor can be disposed on an inner surface of the first portion proximate the first end of the first portion and may be operatively connected to the controller. The controller can be configured to compare the second pressure signal to a vibration threshold and can cause the vibration motor to activate when the second pressure signal exceeds the vibration threshold.

In certain embodiments there can be a hand-like structure hingedly coupled to the coupling portion. At least one digit-like structure may include a plurality of finger digits including a thumb, a first finger digit, a second finger digit, a third finger digit, and a fourth finger digit, wherein each of the respective finger digits can be hingedly connected to and may extend from the first portion at an angle relative to the axis.

In certain embodiments the prosthetic can have a plurality of finger digit sensors, each finger digit sensor can be disposed at a tip of a respective finger digit. The controller can be configured to receive a respective second pressure signal from each of the plurality of finger digit sensors indicative of pressure on the respective finger digit. The controller can be configured to compare each respective second pressure signal to the vibration threshold and can cause the vibration motor to activate when any of the respective second pressure signals exceed the vibration threshold.

In certain embodiments the controller can be configured to convert the second pressure signals to voltage signals and the vibration threshold can be a voltage threshold.

In certain embodiments the vibration motor can be disposed on the inner surface of the first portion proximate the first end of the first portion and at a location configured to contact the residual limb of the user with the user wearing the prosthetic to provide an indication of touch to the user upon activation of the vibration motor.

In accordance with at least one aspect of this disclosure, the prosthetic may be configured for a lower leg. The prosthetic may have a tibial structure that can be operatively coupled to the coupling portion. The prosthetic may further have a foot portion that can be operatively connected to the tibial structure. The foot portion may have a foot sensor that can be operatively connected to the controller. The controller can be configured to receive a second pressure signal from the foot sensor indicative of pressure on the foot. The controller can be configured to compare the second pressure signal to a vibration threshold and may cause the vibration motor to activate when the second pressure signal exceeds the vibration threshold.

In certain embodiments the vibration motor can be disposed on the inner surface of the first portion at a location configured to contact the residual limb of the user with the user wearing the prosthetic to provide an indication of touch to the user upon activation of the vibration motor.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 is a perspective view of an embodiment of a prosthetic in accordance with this disclosure, showing the prosthetic in use by a wearer, where the prosthetic is configured as an upper limb prosthetic;

FIG. 2 is an exploded perspective view of the prosthetic of FIG. 1, showing a second portion exploded from a first portion; and

FIG. 3 is a perspective view of another embodiment of the prosthetic in accordance with this disclosure, where the prosthetic is configured as a lower limb prosthetic.

### DETAILED DESCRIPTION

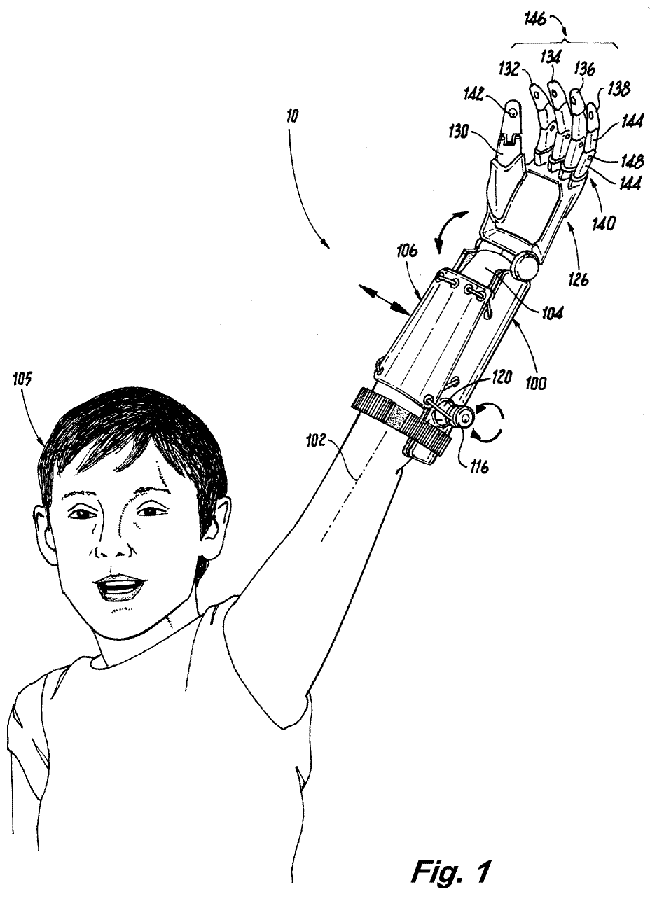

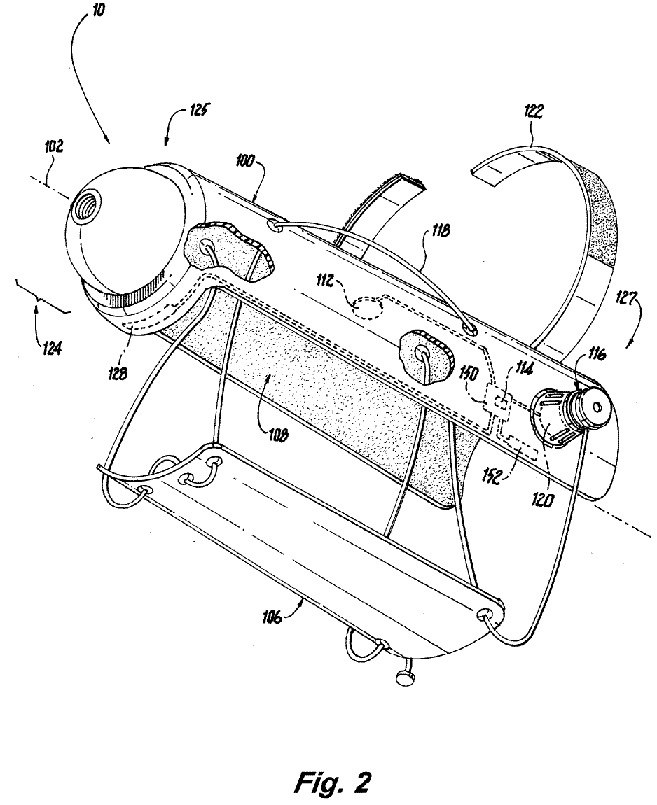

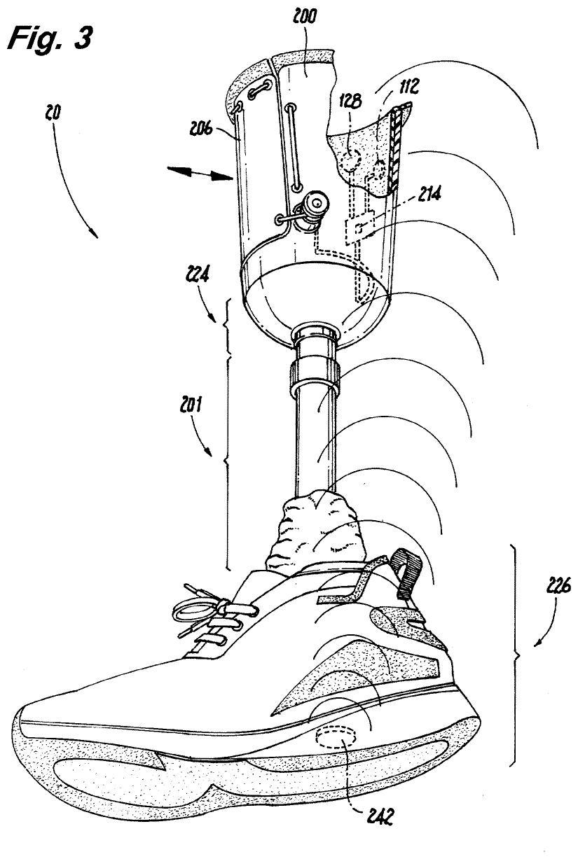

Reference will now be made to the drawings wherein like reference numerals identify similar structural features or aspects of the subject disclosure. For purposes of explanation and illustration, and not limitation, an illustrative view of an embodiment of a prosthetic in accordance with the disclosure is shown in FIG. 2 and is designated generally by reference character 10. Other embodiments and/or aspects of this disclosure are shown in FIGS. 1 and 3.

In accordance with at least one aspect of this disclosure, FIGS. 1 and 2 show an embodiment of a prosthetic. As shown, the prosthetic 10 includes a first portion 100 that extends along an axis 102. The first portion 100 is configured to receive and partially surround a residual limb 104 of a user 105. A second portion 106 is moveably connected to the first portion 100, wherein the first portion 100 and the second portion 106 come together to define an interior space 108 for supporting the prosthetic appendance 10 on the residual limb 104 of the user 105. The second portion 106 is moveably connected to the first portion 100 such that it can move in a perpendicular or radial direction relative to the axis 102. The movement of the second portion 106 allows for adjustment of the volume of the interior space 108 to accommodate radial growth of the residual limb 104, which may occur due to environment, ambient or body temperature, swelling, and/or shrinkage.

A pressure sensor 112 can be disposed on an inner surface 108 of the first portion 100 within the interior space. A controller 114 is operatively connected to the sensor 112 to receive a first pressure signal from the sensor 112 indicative of a pressure of the residual limb 104 on the inner surface 108 of the first portion. The controller 114 can be configured to adjust the second portion 106 relative to the first portion 100 based on the pressure signal received from the sensor 112 to expand or retract the volume of the interior space 108 of the first portion 100 to accommodate radial growth and shrinkage of the residual limb 104 relative to the axis 102. Thus, the prosthetic 10 as described herein provides for automatic adjustment to the size of the prosthetic 10 to improve the overall comfort and functionality of the prosthetic 10 to the user 105.

In certain embodiments, the controller 114 can be configured to convert the pressure signal to volts and compare the voltage reading to a voltage threshold. The controller 114 can then cause the second portion 106 to move relative to the first portion 100 to expand the interior volume if the voltage reading is above a first threshold and retract the volume if the voltage reading is below a second threshold. In certain embodiments, the first threshold can be about 700 volts or about 750 volts or about 800 volts. In certain embodiments, the second threshold can be about 450 volts or about 400 volts or about 350 volts. This means that when the first pressure signal, converted to volts, is greater than about 700 volts to about 800 volts, the second portion 106 will move outward, to expand the volume of the first portion 100 and loosening the prosthetic 10 on the user 105. When the first pressure signal, converted to volts, is less than about 350 volts to about 450 volts, the second portion 106 will move inwards, reducing the volume and tightening the prosthetic 10. When the first pressure signal is between about 400 volts and about 800 volts, the controller 114 will not cause the second portion 106 to move.

In certain embodiments, such as more clearly seen in FIG. 2, a spool 116 can be operatively connected to the first portion 100. The spool 116 can have a filament 118 wound thereon, which connects the second portion 106 to the first portion 100. The filament 118 may be made of plastic, fiber, or a similar material. A motor 120 can be operatively connected to the spool 116 to drive the spool 116, e.g., in response to instruction from the controller 114. For example, the controller 114 can be configured to cause the motor 120 to rotate the spool 116 in a first direction to unwind the spool 116 to expand the volume of the interior space 108 of the first portion 100 when the first pressure signal exceeds the first threshold (e.g., indicating the prosthetic 10 is too tight). Additionally, the controller 114 can be configured to cause the motor 120 to rotate the spool 116 in a second direction to wind the spool 116 to reduce the volume of the interior space 108 of the first portion 100 when the first pressure signal is below the second threshold (e.g., indicating the prosthetic 10 is too loose). The controller 114 will cause the motor 120 to rotate until the pressure signal from the sensor 112 is within the stationary range (e.g., between about 350 volts and 700 volts).

In certain embodiments, the controller 114 can be configured to cause the motor 120 to rotate the spool 116 to a specific position, resulting in a rotation proportional to the voltage signal. For example, the controller can be configured to calculate a delta between the pressure signal and the first threshold and the second threshold. If the delta between the pressure signal and the first threshold is above a first delta threshold, the controller will cause the motor 120 to rotate proportional to the delta. Accordingly, if the delta between the pressure signal and the first threshold is very high, this indicates the prosthetic is very tight, and the controller 114 will cause the motor 120 to rotate the spool 116 more than if the delta were lower. A small delta between the pressure signal and the first threshold indicates the prosthetic is not very tight, but tight enough to be uncomfortable. In this case, the controller 114 will cause the motor 120 to rotate the spool 116 less than the instance where the delta is higher. The same is true regarding the delta calculated between the pressure signal and the second threshold. A high delta between the pressure signal relative to the second threshold means the prosthetic is very loose, thus the controller 114 will cause the motor 120 to rotate the spool 116 more to tighten the prosthetic.

The motor 120 and spool 116 can be configured to rotate between 20 degrees and 120 degrees, assuming 90 degrees is a starting position for the motor 120. This rotational range provides for about 1-1.5 cm of radial growth of the residual limb 104, which is the typical amount of growth for a child's upper limb. In certain embodiments, the motor 120 and spool 116 can be configured to greater rotation as needed or desired to accommodate more or less growth, depending on the needs of the particular wearer.

In certain embodiments the prosthetic 10 can include a coupling portion 124 defined at a first end 125 of the first portion 100 which may be configured and adapted to receive and couple an appendage 126 of the first portion 100. In certain embodiments, like shown in FIG. 1 for example, the prosthetic 10 can be configured for use on an upper limb, and the appendage 126 can be a hand-like structure 126 coupled to the coupling portion 124. While a hand-like appendage 126 is shown in FIG. 1, it should be appreciated by those having ordinary skill the art in view of this disclosure that any suitable appendage can be coupled to the prosthetic, for example, a hook like structure, a claw like structure, a grabbing structure, or the like.

In certain embodiments, the appendage 126 can be configured to provide haptic feedback to the wearer via a vibration motor 128 disposed on an inner surface 108 of the first portion 100, proximate the first end 125 of the first portion 100. The appendage 126 can further include at least one digit-like structure 146 coupled to the base of the appendage 126 which may be coupled to the coupling portion 124. For example, the digit-like structures 146 can include a thumb 130, a first finger digit 132, a second finger digit 134, a third finger digit 136, and a fourth finger digit 138. Each of the respective finger digits 146 can be hingedly 140 connected to and extend from the base of the appendage 126 at an angle relative to the axis 102, such as best seen in FIG. 1. In certain embodiments one or more of the finger digits 146 can have a respective finger digit sensor 142. Each finger digit sensor 142 can be disposed at a tip of the respective finger digit 146.

The controller 114 can be configured to receive a respective second pressure signal from each of the plurality of finger digit sensors 142 indicative of pressure on the respective finger digit 146. The controller 114 can be configured to compare each respective second pressure signal to a vibration threshold and can cause the vibration motor 128 to activate when any of the respective second pressure signals exceed the vibration threshold. The controller 114 can be configured to convert the second pressure signals to voltage signals and the vibration threshold can be a voltage threshold. In certain embodiments, the vibration threshold can be between about 50 volts and about 150 volts.

In certain embodiments, the controller can cause the vibration motor 128 to activate to cause different sensations based on the voltage signal. For example, the voltage threshold can be a low, dull sensation threshold at about 50 volts to about 100 volts, wherein if the second pressure signal exceeds 50 volts but is less than 100 volts, the controller 114 can cause the vibration motor 128 to generate a low, dull vibration. The voltage threshold can be a slight sensation threshold at about 100 volts to about 150 volts, wherein if the second pressure signal exceeds 100 volts but is less than 150 volts, the controller 114 can cause the vibration motor 128 to generate a slight vibration, greater than the low, dull vibration. The voltage threshold can be a significant sensation threshold between about 150 volts and 200 volts, wherein if the second pressure signal exceeds 150 volts but is less than 200 volts, the controller 114 can cause the vibration motor 128 to generate a significant vibration, greater than the low, dull vibration and greater than the slight vibration. The voltage threshold can be a forceful sensation threshold at about 150 volts and greater, wherein if the second pressure signal exceeds 150 volts, the controller 114 can cause the vibration motor 128 to generate a significant vibration, greater than the low, dull vibration and greater than the slight vibration. In certain embodiments, the controller 114 can be configured to compare the voltage signal to all three of the thresholds to provide varying haptic feedback to the wearer based on how hard they are touching a surface, to recreate the feeling of touch.

The digit-like structures 146 can be attached to the base of the appendage 126 at a respective hinge 140. Each digit-like structure 146 can also have multiple segments 144 connected to one another via respective secondary joints 148. In certain embodiments, the digit-like structures 146 and respective segments 144 can be connected via filament (e.g. strung together) similar to a ligament so that the digit-like structures 146 can flex and extend to allow for a grabbing and releasing motion. In certain embodiments, the digit-like structures 146 can be connected together in a Whipple tree configuration, where a base of the tree is included in base of the appendage 126 such that movement of the appendage 126 causes movement in the digit-like structures 146.

Still with reference to FIG. 2, in certain embodiments the prosthetic 10 can further include a strap 122 that can be operatively connected to a second 127 end of the first portion 100. In use, the residual limb 104 is inserted into the interior space 108 and the strap 122 can be used to secure the prosthetic 10 to the residual limb 104 if needed or desired. The strap 122 may be adjustable to ensure the prosthetic 10 remains on the residual limb 104, for example when the controller 114 is tightening the second portion 106 relative to the first portion 100. However, the strap 122 can be an optional feature based on the preference of the user 105.

In certain embodiments, the controller 114 can be included on a circuit board 150 embedded within the first portion 100 or included on top of the first portion 100. The controller 114, sensors 112 and 142, and motors 120 and 128 can be connected to one another in any suitable manner, such as via hard wired or wireless connections. In certain embodiments, one or more Bluetooth controllers can be utilized for communication between the controller 114 and the sensors 112 and 142 and/or motors 120 and 128. In certain embodiments, the prosthetic 10 can further include a battery 152 operatively connected to power the electronics onboard the prosthetic 10. In certain embodiments battery can be a rechargeable battery.

Any one or more portions of the first portion 100 and/or second potion 106 and/or appendage 126 can be made out of lightweight durable material, such as a plastic. In certain embodiments, the plastic material can be a 3D printable material to allow for complex shapes and accurate connection points on a small scale, such as between the digit-like structures 146 and the first portion 100. In certain embodiments, the material can be or include a water-soluble printing material configured to provide smooth and flexible characteristics to the prosthetic 10 for maximizing comfort and range of movement.

Turning now to FIG. 3, another embodiment of the prosthetic is shown, which is configured and adapted for use on a lower leg. The prosthetic 20 can be similar to prosthetic 10 and can have similar components and features with respect to prosthetic 10. For brevity, the description of common elements that have been described above for prosthetic 10 will not be repeated with respect to prosthetic appendage 20 as shown in FIG. 3.

As shown in this example, a tibial structure 201 is operatively connected to the coupling portion 224 and a foot portion 226 is operatively connected to the tibial structure 201. In certain embodiments, the foot portion 226 can be a realistic or traditional looking foot or can be a running blade or the like. The controller 214 is configured to cause the second portion 206 to move relative to the first portion 200 to expand or retract the volume with radial expansion or shrinkage of the user's lower residual limb, similar to that discussed above with respect to the prosthetic 10. Also, in certain embodiments, the foot portion 226 comprises a foot sensor 242 operatively connected to the controller 214. Like the digit sensors 142 discussed above, the foot sensor 242 is configured to cause the controller 214 to activate the vibration motor 228 when the pressure on the sensor 242 exceeds the vibration threshold to provide haptic feedback to the wearer when the foot 226 is in contact with the ground, for example. While the sensor 242 is shown in the heel region of the foot portion 226, it should be appreciated that the sensor can be placed in any suitable location, such as a toe region, which could help the user learn to walk with the prosthetic in a more fluid and natural movement since haptic feedback would be provided when the foot is rolled from heel to toe, for example.

Congenital limb differences and limb losses from trauma are significant health conditions. Many individuals do not have proper prosthetics because the prosthetics can be both prohibitively expensive and can contain many issues around their functionality, sizing, and comfort, making them an inadequate option for certain prosthetic users, such as children.

Embodiments of the prosthetic appendage discussed herein addresses many of the challenges and shortcomings of conventional prosthetics. In certain embodiments, the prosthetic uses 3D-printed and cost-conscious materials to make the prosthetic more affordable and durable. Embodiments can also automatically adjust the size of the prosthetic depending on the user's limb changes (e.g., due to diurnal fluctuation), eliminating issues such as skin irritation, the need to be constantly replaced, hyperhidrosis, and discomfort due to tightness. Also, in certain embodiments, the prosthetic can utilize a haptic sensor-vibration motor circuit to return haptic sensation to the user. This replicates sense of touch when the user engages in physical activity, such as grabbing an object or stepping on the ground. Embodiments makes using a prosthetic much easier, especially for children, while also making the prosthetic less prone to damage because users will be aware of when the prosthetic is having physical contact. As a result of these novel and non-obvious features, embodiments of the prosthetic increase the useable period of the prosthetic significantly.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. A prosthetic, comprising:
a first portion extending along an axis configured to receive and partially surround a residual limb of a user;
a second portion moveably connected to the first portion, wherein the first portion and the second portion come together to define an interior space for supporting the prosthetic appendance on the residual limb of the user;
a sensor disposed on an inner surface of the first portion and within the interior space; and
a controller operatively connected to the sensor to receive a first pressure signal from the sensor indicative of a pressure of the residual limb on the inner surface of the first portion, the controller being configured to adjust the second portion relative to the first portion based on the pressure signal received from the sensor to expand or retract a volume of the interior space of the first portion to accommodate radial growth and shrinkage of the residual limb relative to the an axis.

2. The prosthetic of claim 1, further comprising:
a spool having a filament wound thereon, wherein the second portion is connected to the first portion by the filament; and
a motor operatively connected to the spool to drive the spool, wherein the controller is configured to cause the motor to rotate the spool in a first direction to unwind the spool to expand the volume of the interior space of the first portion when the first pressure signal from the sensor is above a first threshold, and wherein the controller is configured to cause the motor to rotate the spool in a second direction to wind the spool to retract the volume of the interior space of the first portion when the first pressure signal from the sensor is below a second threshold.

3. The prosthetic of claim 1, wherein the controller is configured to convert the pressure signal to a voltage signal, and wherein the first threshold is a first voltage threshold and wherein the second threshold is a second voltage threshold different from the first voltage threshold.

4. The prosthetic of claim 2, further comprising, a coupling portion defined at a first end of the first portion configured and adapted to receive and couple an appendage to the first portion.

5. The prosthetic of claim 4, wherein the appendage includes at least one digit-like structure, and wherein the at least one digit-like structure is hingedly coupled to the coupling portion.

6. The prosthetic of claim 5, further comprising:
a digit sensor disposed on a tip of the at least one digit-like structure operatively connected to the controller, wherein the controller is configured to receive a second pressure signal from the digit sensor indicative of a pressure on the at least one digit-like structure; and
a vibration motor disposed on an inner surface of the first portion proximate the first end of the first portion and operatively connected to the controller, wherein the controller is configured to compare the second pressure signal to a vibration threshold and cause the vibration motor to activate when the second pressure signal exceeds the vibration threshold.

7. The prosthetic of claim 6, wherein the appendage is a hand hingedly coupled to the coupling portion, and wherein the at least one digit-like structure includes a plurality of finger digits including a thumb, a first finger digit, a second finger digit, a third finger digit, and a fourth finger digit, wherein each of the respective finger digits are hingedly connected to and extend from the first portion at an angle relative to the axis.

8. The prosthetic of claim 7, further comprising a plurality of finger digit sensors, each finger digit sensor disposed at a tip of a respective finger digit, wherein the controller is configured to receive a respective second pressure signal from each of the plurality of finger digit sensors indicative of pressure on the respective finger digit, wherein the controller is configured to compare the each respective second pressure signal to the vibration threshold and cause the vibration motor to activate when any of the respective second pressure signals exceeds the vibration threshold.

9. The prosthetic of claim 8, wherein the controller is configured to convert the second pressure signals to voltage signals and wherein the vibration threshold is a voltage threshold.

10. The prosthetic of claim 9, wherein the vibration motor is disposed on the inner surface of the first portion proximate the first end of the first portion and at a location configured to contact the residual limb of the user with the user wearing the prosthetic to provide an indication of touch to the user upon activation of the vibration motor.

11. The prosthetic of claim 4, wherein the appendage is a lower leg, and further comprising:
a tibial structure operatively coupled to the coupling portion;
a foot portion operatively connected to the tibial structure; and
a foot sensor operatively connected to the controller, wherein the controller is configured to receive a second pressure signal from the foot sensor indicative of pressure on the foot, wherein the controller is configured to compare the second pressure signal to a vibration threshold and cause the vibration motor to activate when the second pressure signal exceeds the vibration threshold.

12. The prosthetic of claim 11, wherein the vibration motor is disposed on the inner surface of the first portion at a location configured to contact the residual limb of the user with the user wearing the prosthetic to provide an indication of touch to the user upon activation of the vibration motor.

13. The prosthetic of claim 2, further comprising a circuit board, wherein the controller and the sensor are operatively connected to the circuit board.

14. The prosthetic of claim 13, wherein the circuit board is embedded within the first portion.

15. The prosthetic of claim 13, further comprising a power source operatively connected to the circuit board, configured to power the controller and the sensor.

16. The prosthetic of claim 15, wherein the power source includes a rechargeable battery.
